(19) United States
(12) Patent Application Publication
(10) Pub. No.: US 2023/0233795 A1
(43) Pub. Date: Jul. 27, 2023

(54) SYSTEMS AND METHODS FOR ALTERING NEURAL RESPONSE USING SENSORY INPUT REDUCTION

(71) Applicant: NeuroNest Corp., ALAMO, CA (US)

(72) Inventors: Sharon Seul Evans, Alamo, CA (US); Paul Krebs, Alamo, CA (US)

(73) Assignee: NeuroNest Corp., Alamo, CA (US)

(21) Appl. No.: 18/101,805

(22) Filed: Jan. 26, 2023

Related U.S. Application Data
(60) Provisional application No. 63/303,707, filed on Jan. 27, 2022.

(51) Int. Cl. A61M 21/02; A47G 9/10

(52) U.S. Cl. CPC A61M 21/02; A47G 9/10; A61M 2021/0022

(57) ABSTRACT

A therapeutic nesting apparatus is disclosed for a treating sensory-related, neuropsychological conditions by altering neural response to reduce sensory input and improve biological, neurological, and psychological performance. The apparatus provides for a body to lie in a natural position creating a cocoon-like sensation that allows the brain to alter neural response. The nesting apparatus allows the user's head to be relaxed for open airways and minimize strain on the neck. The lower portion of the nesting apparatus may support the legs in a gently bent position to allow the feet to be on or off the ground depending on the user's preference. The sides of the nesting apparatus are designed to hold the user's arms by their side to give a hug-like sensation as well as allowing the arms to be free.

### CROSS-REFERENCE TO RELATED APPLICATIONS

This application claims the benefit of U.S. Provisional Patent Application No. 63/303,707, filed Jan. 27, 2022, the content of which is hereby incorporated by reference in its entirety as if set forth herein.

### FIELD OF TECHNOLOGY

The present invention relates to systems and methods for treating sensory-related, neuropsychological conditions, more particularly to methods and apparatuses for altering neural response to reduce sensory input and improve biological, neurological, and psychological performance.

### BACKGROUND

Neuropsychological conditions with a sensory-processing related component, such as autism spectrum disorder, post-traumatic stress disorder (PTSD), and anxiety, affect millions of persons worldwide. In terms of PTSD alone, it is estimated that 83% of the population has been exposed to severe and potentially traumatic events, with 8% of those affected developing a diagnosis of PTSD. Additionally, 14% of the population meets diagnostic criteria for an anxiety disorder at any particular time, and 1 in 54 children meet criteria for autism spectrum disorder. Current pharmacological and behavioral treatments for these conditions are not useful for many people, do not assist a large percentage of those diagnosed, and can have serious iatrogenic side effects.

When treating anxiety disorders and PTSD, research shows that cognitive-behavioral and exposure-based interventions can be effective, but only lead to clinically significant loss of diagnosis in about 51-66% of cases. Additionally, these treatments are limited by their acceptability in terms of time, cost, and convenience. Therapies for PTSD, for instance, rely on exposure to traumatic experiences, which can be difficult to tolerate and result in treatment dropout rates of up to 65%. Behavioral therapies for autism, such as applied behavioral analysis, have failed to show improvements in general symptoms, language, adaptive behavior, and daily living skills. Interventions such as squeeze machines/compression are costly, inaccessible, and are difficult to use with children. Other postural interventions, such as yoga, are often not feasible or acceptable to many people as well.

Pharmacological treatments for PTSD, anxiety, and autism can assist in symptom management but do not cure these conditions. The Institute of Medicine concluded that efficacy of SSRIs for PTSD is at best suggestive. In terms of anxiety, response rates to medications remain low with only about 30-36% of people achieving diagnostic remission. Further, medications create a number of side effects, including delayed onset of action, loss of sexual drive, and changes in body weight. Of significant concern, the majority of patients with anxiety and PTSD are treated with benzodiazepines, which are characterized by a number of problems including dependency and central nervous system (CNS) depression, resulting in fatigue, dizziness, and impaired motor and cognitive functions, especially in elderly patients. Regarding autism-spectrum disorders, a range of medications are used to mitigate symptoms. Ritalin and Adderall are commonly suggested for hyperactivity, with antipsychotic drugs such as risperidone and aripiprazole, prescribed for irritability and aggression. These drugs have significant negative side effects and merely ameliorate symptoms rather than treat the core neurodevelopmental condition.

Of additional concern, serious barriers exist for people accessing and accepting behavioral and pharmacological treatments for these sensory-related conditions. Access to treatment is dependent on a number of circumstances largely out of people's control including availability of treatment facilities and professionals, time to attend appointments, health insurance, family support, other children, and employment. For those with autism, adults age out of eligibility and insurance coverage for most behavioral treatment programs. Even with access, the majority of people with these conditions are untreated due to unease with engaging in current treatments. For instance, in a survey of people experiencing PTSD, more than one-third (35%) reported that they were not emotionally ready for treatment and 52% indicated that they believed they did not need treatment. Stigma about treatment is also a key concern, with people noting a sentiment of weakness in seeking treatment and difficulty asking for help because of pride. The latter is of particular relevance among certain cultural groups and the military. Treatments for sensory-related conditions that are low cost, accessible, private, and effective are not yet available and are urgently needed.

### SUMMARY

Aspects of the present invention include a nesting methods and apparatuses to place a body in a natural position to create multiple pathways of sensory experience. Aspects of the present disclosure provide systems and methods for creating a cocoon-like sensation in a user that allows the brain to alter neural response and/or obtain homeostasis. Aspects of the nesting apparatus allow the user's head to be relaxed for open airways and to minimize strain on the neck. A pillow may support the neck and a leg support may provide a gently bent position of the user's legs to allow the feet to be on or off the ground depending on the user's preference. Such a feature may also relieve pressure off the user's back. The sides of the nesting apparatus are designed to hold the user's arms by their side to give a hug-like sensation while allowing the arms to be free.

According to certain aspects of the present disclosure a nesting apparatus for supporting a user is provided. The nesting apparatus may include a platform, a first side support, a second side support, and a headrest coupled to the platform. A base pad may be disposed on the platform between the first and second side supports and the headrest. First and second side pads may be respectively coupled to an inside surface of the first and second side supports. A headrest pad may be coupled to an inside surface of the headrest. The headrest pad, first and second side pads and base pads may be configured to apply a compressible pressure on a portion of the user's body.

According to another aspect, a method of sensory intervention is disclosed. The method may include applying a first compressible pressure on a first portion of a user's body by a base pad supported by a platform. A second compressible pressure may be applied on a second portion of a user's body by first side pad. A third compressible pressure may be applied on a third portion of a user's body by second side pad. A fourth compressible pressure may be applied on a head and neck of a user's body by a pillow. The first, second, third, and forth compressible pressures may reduce sensory input on the user's body.

According to another aspect of the present disclosure, a therapeutic device for sensory based interventions is disclosed. The device may include a platform, a first padded side support, a second padded side support, and a padded headrest slidably coupled to the platform. A base pad may be disposed on the platform between the first and second padded side supports and the headrest. A headrest pad may be disposed at a first end of the base pad. A leg support may be disposed at a second end of the base pad. The headrest pad, first and second padded side supports, the base pad, and leg support may be configured to apply a compressible pressure on a portion of the user's body, thereby altering neural responses in the user.

### BRIEF DESCRIPTION OF THE DRAWINGS

The features, nature, and advantages of the present disclosure will become more apparent from the detailed description set forth below when taken in conjunction with the drawings in which like reference characters identify correspondingly throughout.

FIG. 1A illustrates a side view of a nesting apparatus, according to one aspect of the present disclosure.

FIG. 1B illustrates a perspective view of the nesting apparatus of FIG. 1A, according to one aspect of the present disclosure.

FIG. 2A illustrates a perspective view of a nesting apparatus, according to one aspect of the present disclosure.

FIG. 2B illustrates a top view of the nesting apparatus of FIG. 2A, according to one aspect of the present disclosure.

FIG. 2C illustrates a side view of the nesting apparatus of FIG. 2A, according to one aspect of the present disclosure.

FIG. 2D illustrates a side view of the nesting apparatus of FIG. 2A in use, according to one aspect of the present disclosure.

FIG. 2E illustrates an exploded view of the nesting apparatus of FIG. 2A according to one aspect of the present disclosure.

FIG. 3A illustrates a top view of a nesting apparatus, according to one aspect of the present disclosure.

FIG. 3B illustrates a perspective view of the nesting apparatus of FIG. 3A according to one aspect of the present disclosure.

FIG. 3C illustrates a partially exploded view of the nesting apparatus of FIG. 3A according to one aspect of the present disclosure.

FIG. 4 is as flow chart depicting a method according to one aspect of the present disclosure.

### DETAILED DESCRIPTION

Aspects of the invention will now be described more fully hereinafter with reference to the accompanying figures, in which preferred embodiments are shown. The foregoing may, however, be embodied in many different forms and should not be construed as limited to the illustrated embodiments set forth herein.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” “substantially,” or the like, when accompanying a numerical value or direction are to be construed as indicating a deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples, or exemplary language (“e.g.,” “such as,” or the like) provided herein, is intended merely to better illuminate the embodiments and does not pose a limitation on the scope of the embodiments. No language in the specification should be construed as indicating any unclaimed element as essential to the practice of the embodiments.

In the following description, it is understood that terms such as “first,” “second,” “third,” “above,” “below,” and the like, are words of convenience and are not to be construed as limiting terms unless expressly state otherwise.

Aspects of the present disclosure include nesting methods and apparatuses by which a person may be positioned to alter neural response and reduce sensory input, thereby improving biological, neurological, and psychological performance.

Generally, aspects of the present disclosure provide a nesting apparatus to provide, or place a user in, an open posture for creating a parasympathetic autonomic response, including, but not limited to, posturing a user with shoulders back, legs supported, and neck in a relaxed state. A user may be positioned, or position themselves, in or on the nesting apparatus as described herein to reduce sensory input on or around the user's body, thereby providing a therapeutic, cocoon-like sensation allowing the brain alter neural response

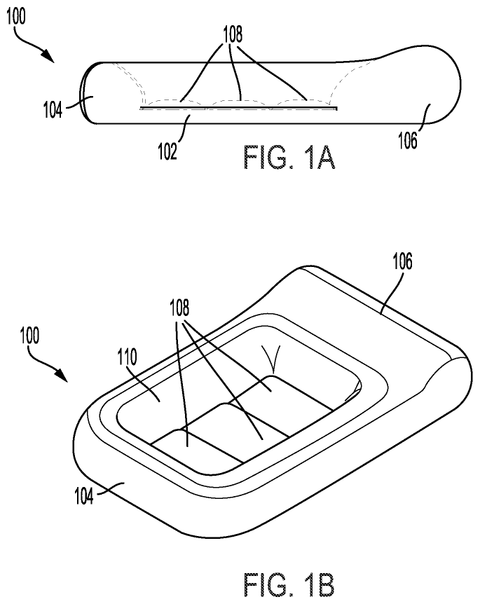

Referring now to FIGS. 1A-1B, a nesting apparatus 100 is provided according to one or more aspects of the present disclosure. According to one aspect the nesting apparatus 100 may include a base portion 102, a headrest 104 and a leg rest 106. The base portion may include a mat, cushion, or other supporting or compressible material adapted to absorb applied weight of the user. The base portion 102 may further include or define one or more individual chambers 108 having support memory cooling gel or other stuffing configured to prevent overheating and facilitate comfort and body support. According to one aspect, the chambers 108 may be inflatable or expandable to increase pressure and structural support. The chambers 108 may inflate or deflate by manual pump to a desired comfort level. The inflated chambers 108 may be adapted to cradle the user's form to provide a sense of weightlessness or pillow-like comfort through a compressible pressure, further augmenting the cocoon-like sensation.

The headrest 104 may be designed to cradle and support the neck as to create easy and natural breath for individuals while simultaneously not blocking one's peripheral view. A leg support (106) may be designed for support and comfort for the legs to relieve stress on the back and/or support the feet where they either can dangle or be comfortably placed on the ground for a secure feeling. The leg support 106 may be adapted to fit underneath the user's knees, further alleviating stress on the user's back or spine. The cushion of the leg support 106, according to one aspect may be designed with cooling gel foam to maximize variants on weight and support.

According to one aspect, the nesting apparatus 100 may vary in size and shape to accommodate users of varying heights, and weights. Outside portions, or walls 110, may be designed to create a firm yet soft cocoon-like sensation where, under the weight of the user's body, the wall tops slightly envelope the user. The walls 110 may be further adapted to create be even firmer structure that will cradle and support the user.

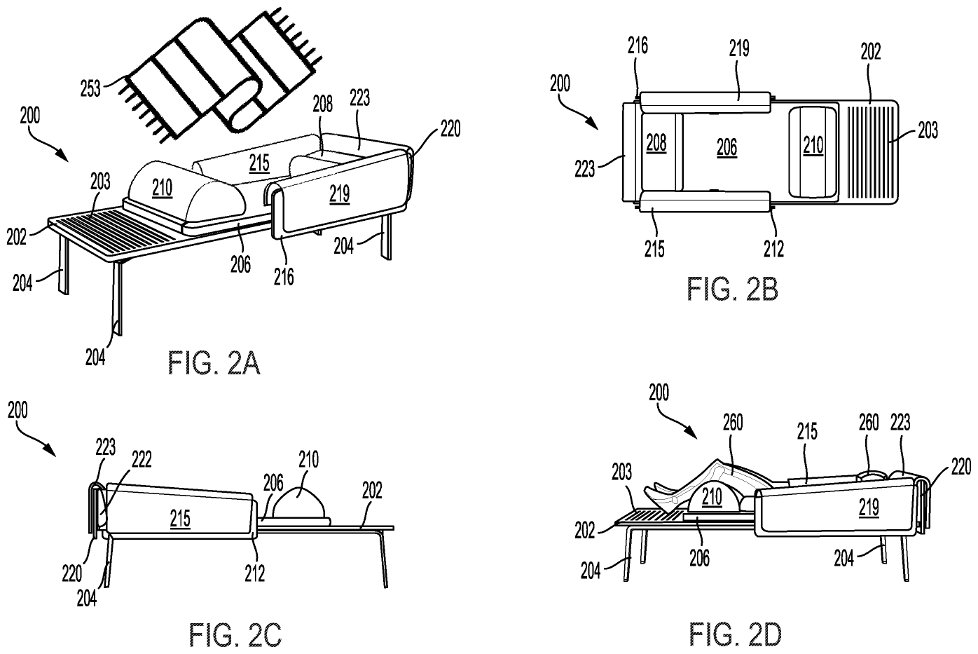

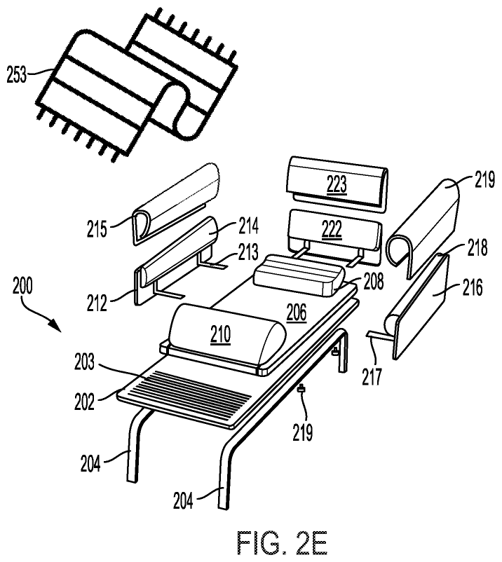

With reference now to FIGS. 2A-2E, another nesting apparatus 200 is depicted according to aspects of the present disclosure. The nesting apparatus 200 may include a platform 202 elevated and supported by legs 204. The legs 204 may include four separate legs or, preferably, include two U-shaped bars extending the length of, and disposed near the long-side edges, of the platform 202. The platform 202 may be formed from wood, plywood, medium-density fiberboard, plastic, or any other suitable firm material. The platform may also feature recesses or slots 203 for the placement of the user's feet. The slots 203 may serve to provide a friction against the feet, thereby preventing the user's feet from continually slipping when in use. The slots 203 also provide a gripping surface for the user's feet to assist entering and exit the nesting apparatus.

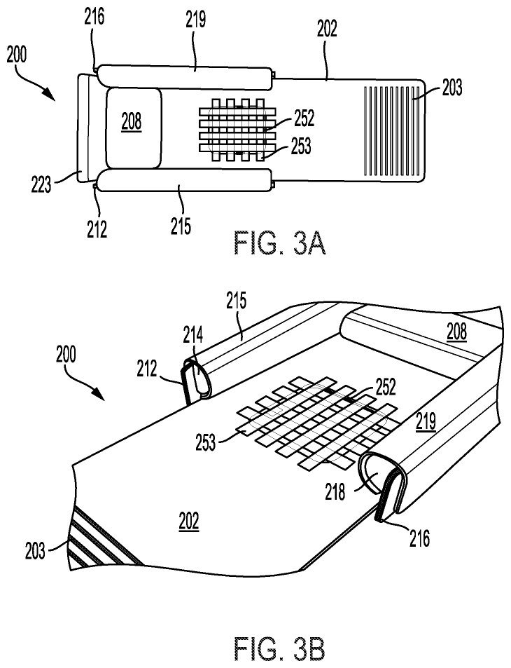

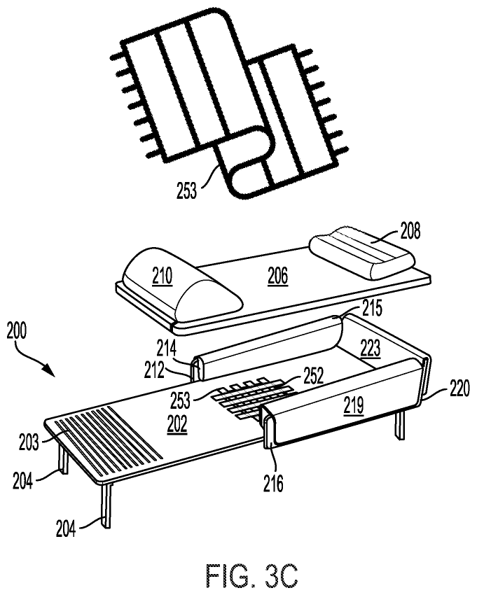

According to another aspect of the present disclosure, as depicted in FIGS. 3A-3C, the platform 202 may include or define a hole 252 in or about the center of the platform 202, covered by a netting 254. The netting may include one or more straps, bands, or other flexible materials that yield a limited or small amount under the weight of the user. The hole 252 and netting 254, when covered by a base pad 206, may alleviate pressure on the user's backside while simultaneously providing additional cradling and sensory comfort to the user.

Referring now to FIGS. 2A-3C, the nesting apparatus 200 may include one or more side supports, such as first and second side supports 212, 216. The side supports 212, 216 may be coupled to the platform 202 using brackets 213, 217, respectively, or other coupling structures. The side supports 212, 216, when assembled may be substantially perpendicular to the platform 202. The brackets 213, 217 may be coupled to the platform in an adjustable, sliding arrangement. The adjustability of the side supports 212, 216 allow for the formation of a nesting space of varying sizes and pressure. For example, the brackets 213, 217 may be coupled to one or more tracks or other receptive device the underside of the platform 202. An adjustable fastener, such as knob 219, screw, or other fastener may be used to selectively secure the bracket to the platform, allowing the side supports 212, 216 to slide laterally. When the side supports are in a desired position, the knob may be tightened to secure the side supports 212, 216 in place.

The side supports 212, 216 may include side pads 214, 218 affixed or disposed on the inside surface of the side supports 212, 216. The side pads 214, 218 may include or form cushioning or padding adapted to apply a compressible pressure to the side of the user's body when positioned in the nesting apparatus 200. The side pads 214, 218, according to one aspect may have an adjustable level of cushioning or stuffing to accommodate different desired compressible pressures or user body sizes. Each side support 212, 216 may also include side covers 215, 219 disposed over the side pads 214, 218, further enhancing the cushioning effect on the user's sides. According to one aspect, the side covers 215, 219 may include or form a pocket area where the side supports 212, 216 meet the platform. Such a configuration and design further enhance a burrowing, cocoon-like sensation. The side supports 212, 216 may also be designed so the height obscures the user's vision as little as possible. The side height and width of the side pads 214, 218 may be designed to be snug such that the user's arms and hands can be held close to the body.

A head rest 220 may be coupled to the platform 202 at one end, using for example, brackets 221 or the like. Similarly to the side supports 212, 216, the headrest may be slidably coupled to the platform 202 to adjust the size and shape of the nesting area. The headrest 220 may, like the side supports 212, 216, include a headrest pad 222 and headrest cover 223. The headrest 220 provides a third wall to the nesting apparatus 200 further enveloping the user, when in position.

A base pad 206 may be disposed on the platform 202 providing a cushioning support for the user. The base pad 206 may be formed from a memory foam, gel, or other cushioning material. According to one aspect, the base pad may include a warming or cooling (electric or non-electric) feature to regulate or control the temperature of the user's body. The base pad may be configured to provide cushion a user's back such that the user feels little or no pressure. A pillow 208 may be disposed near the headrest 220 and be sized and shaped to accommodate the user's head and neck. The pillow 208 of the nesting apparatus 200 may include bottom and side portions that allow the head to be cradled such that peripheral vision is not impaired and the neck has open airways and is supported with the rest of the head. It may be vital that persons with sensory disorders be able to have minimal to no visual interference in order to feel safe. Accordingly, the pillow may be sized and shaped, in conjunction with the height of the side supports 212, 216 to maximize the user's peripheral vision. According to one aspect, the pillow 208 may be contoured to optimally cradle the user's neck and head, thereby removing stress and strain. A leg rest 210 may be disposed near the end of the base pad 206 opposite the headrest 220. The leg rest 210 may be sized and shaped to support the user's legs, behind the knees, placing the user in an optimal, reduced strain position, as shown in FIG. 2D.

According to one aspect, the nesting apparatus 200 may include a control and mechanical actuation to create or change desired level of compressible pressures, depending on the user or type of therapy being applied. According to another aspect, the nesting device may include one or more sensors to detect applied pressures on one or more portions of the body. The sensors and a controller may be configured to automatically adjust or maintain compressible pressures applied to the user. For example, one ore more sensors may be in communication with a controller wo ensure an even and equal pressure is applied by the various components of the nesting apparatus 200. The nesting device 200 may also include grounding copper pad coupled to the platform 202 or other components.

In operation and treatment, the nesting apparatus 200 works by creating multiple pathways of sensory experience including but not limited to, postural support, pressure, compression, temperature, and relaxation. The user-contacting pads and covers, such as the base pad 206, side covers 215, 219, pillow 208, and headrest cover 223, may be adapted to cradle the user's form to provide a sense of weightlessness or pillow-like comfort through a compressible pressure, further augmenting the cocoon-like sensation. Application of a weighted blanket 253 over the user may fully encapsulate the user, providing a fully nested environment. According to one aspect, the weighted blanket may include a warming or cooling feature (electric or non-electric) to regulate or control the user's body temperature. The nesting apparatus 200 may create a cocoon or nest-like sensation allowing the user's body to create new neural responses to improve conditions characterized by sympathetic nervous system activation and/or sensory processing components (e.g., Autism, PTSD, and Anxiety, Insomnia, and Chronic Stress).

Many neurodevelopmental and psychological conditions may be characterized by chronic over-excitation of the limbic system, sensory hyperactivity and sensory integration disruptions. Hyperactivity in these sensory input and processing centers may lead to overload of regulatory systems, such as the frontal lobes and hippocampus. The multiple pathways of sensory intervention created by the nesting apparatus 200 (such as postural support, pressure, compression, temperature) may facilitate an inhibitory response in sensory input and processing centers, thereby reducing negative symptoms such as hypervigilance, sensitivity to sound, avoidance, and aggression associated with these conditions.

The use of compressible pressure is based on sensory integration theory, which proposes that therapeutic interventions may be used to incorporate sensation to affect multi-sensory perception and thereby influence learning and behavior. Aspects of the present disclosure use sensory-based interventions which have traditionally been used to assist people with autism spectrum disorder who experience sensory over-responsivity (SOR) at high rates (56-70%). SOR is also found in the general population at rates of 10-17%.

A number of studies have been conducted supporting the efficacy of pressure. Deep pressure therapies have included weighted garments, swaddling, holding, stroking, hugging, squeezing, and therapeutic brushing. Studies have shown that the use of compressive garments had a significant effect with significantly improved results relating to irritability, hyperactivity, lethargy, postural control in dorsal and profile positions, and motor performance. A squeeze machine (resembling a cattle chute) has also been studied in which deep pressure in a squeeze machine was employed on 12 young people with autism compared with being placed in the machine but without any pressure. Physiological measures (Galvanic Skin Response, GSR) did not show any difference, but a parent rating of anxiety did. A post hoc analysis suggested that those with higher arousal preintervention seemed to benefit more than the lower arousal group. In another study of 23 college students, the squeeze machine was found to feel more relaxing than a control treatment on subjective scoring, although objective measures showed no difference. In a study of 20 children with autism spectrum disorder, neurobiological stress as measured by galvanic skin response and conduct problems were reduced in children who were wearing an inflatable wrap after a three-week intervention period.

Other studies have examined use of pressure for anxiety using a within-subjects design (N=12) finding that weighted blankets reduced signs of dental anxiety in students when measured physiologically. And in a case study using deep touch pressure for comorbid anxiety in bipolar disorder, the patient reported that the techniques were helpful in coping with overwhelming situations in her environment. Clinician-rated functioning also improved over the course of treatment (Sylvia et al., 2014).

According to aspects of the present disclosure, the nesting apparatus 200 creates for the user an open posture with the shoulders back, legs supported, and neck in a relaxed state. Evidence exists that an open posture as compared to a slumped posture is associated with the relaxation response (parasympathetic autonomic response) whereas a slumped or tight posture is associated with the fight or flight response (sympathetic autonomic response). The interactions between perception in the brain and effects on the body are strongly bidirectional such that postural feedback from the body to the limbic system (“bottom up” processing) can induce the relaxation response. Mind-body interactions are mediated by peripheral nerves. Somatic sensory nerves carry tactile, proprioceptive, and pain information from skin, striated muscles, and joints. The vagus (10th cranial nerve) is composed of over 80% sensory (afferent) fibers that relay information regarding peripheral visceral sensation, organ function, and immune-inflammatory status to limbic and cortical centers. Reciprocal vagal pathways support transmission of psychophysiological information from peripheral visceral tissue/organs to the brainstem and upward to the executive homeostatic network (EHN) and back down. Thus, the bidirectional vagal system provides an important pathway through which mind-body therapies may diminish stress-related symptoms encoded in the brain and expressed at the body.

A small but suggestive number of studies exist regarding the intervention potential of these neuroscientific findings. In a high-quality randomized study, an open posture was associated with higher self-esteem, more arousal, better mood, and lower fear and has also been found to be associated with increased accuracy of judgement. Additionally strong evidence from the study of yoga indicates posture results in improved autonomic functioning.

According to another aspect, the nesting apparatus 200 may also be used as a complement to other interventions such as Stress Inoculation Training to assist in the prevention of anxiety and trauma-related symptoms for those who anticipate such situations such as military and first responders. Stress Inoculation Training (SIT) is a cognitive and relaxation-training based intervention that has been found to be effective for a range of conditions including anxiety among parents of children undergoing medical procedures, anxiety disorders, stress, anxiety, and depression among cancer patients, PTSD among sexual assault survivors, and military veterans with PTSD. A key phase of SIT is teaching participants relaxation training techniques to improve coping skills when faced with stressors. Accordingly, the nesting apparatus 200 may be used to assist users in downregulating their nervous systems more efficiently on their own during challenging situations and also to assist in recovery following such events.

The nesting apparatus 200 may reflect an effective way for the human body to be supported and able to achieve a relaxed mindset faster. States of meditation have shown to regenerate and create new neural pathways, but achieving this state takes years. For many people, the thought of touch and being around people is too overwhelming and sometimes the situation that causes anxiety or PTSD is too difficult to address in therapy. The nesting apparatus 200 is designed to allow the senses to calm down or repair allowing individuals to live without fear of triggers that can overwhelm their daily life.

According to one aspect, cooling gel mats may be included in or as the base pad 206 to help regulate body temperature and give compression creating a cocoon/hug sensation to the user. According to other aspect of the present disclosure the nesting apparatus 200 may be combined with or incorporate a number of complimentary therapies to enhance the efficacy of the parasympathetic autonomic response. Those potential therapies may include but are not limited to: a cover/pad to enable magnetic therapies; a vibrating cover/pad designed to pulse at various rates (e.g. the same rate of a meditating monk or a peaceful heart rate); acupressure mats that may supply hundreds of pressure points on the user's back; grounding mats that relieve the stress of electronics; and/or a vibrating magnetic pad that combines the individual benefits described herein.

According to one or more aspects, the cover materials of the base pad 206, side pads 215, 219, headrest cover 223, the headrest 208 and/or leg support 210 may be designed to be removable, machine washable, waterproof, and breathable. The base pad 206 may also be designed to help slippage by including gripping or friction-inducing material on the underside where the base pad 206 engages the platform 202. According to one aspect, variants on the cover materials may include using magnetic and vibrational technology. Such covers/pads may increase the possibility of blood flow and comfort to the individual. The covers/pads may have strategically placed magnets that are concealed in the cover. The magnets may be covered in rubber to prevent rusting when washing. The vibration cover/pad can be powered and/or charged with a standard outlet or battery.

According to one aspect, the material used in the nesting apparatus 200 may include organic cooling shredded memory foam, gel and other stuffing that may be wrapped in a waterproof material to ensure moisture does not ruin the apparatus if the outside material is exposed to water or other liquids. The base pad 106, according to one aspect, may be a much thicker support structure to withstand different amounts of weight comfortably.

According to one aspect, the inner structure of the nesting apparatus, in particular the base pad 206, the side pads 214, 218, side covers 215, 219, headrest pad 222, pillow 208 and leg support 210, may be designed to create a gentle nest-like feeling that will cradle the user's body. According to one aspect, the base pad 206 may be filled with, or include, cooling memory foam or gel to support individually the different parts of the body. The nesting apparatus 200 may be designed to have the capability of having multiple levels of sensory squeezing to allow the individual to have a more immediate feeling of relaxation. The position of the user's legs can be adjusted using the leg support 210 such that the feet can be in the air or on the platform 202 for security. The leg support 210 may be configured to gently support the nesting of the user's legs, which can be important in supporting the body to relieve pressure on the user's back.

According to one aspect, speakers and other audio equipment may be built into the nesting apparatus 200 to assist in reducing the need for a separate headset. Further, the components of nesting apparatus 200 may include one or more vibration features disposed in or coupled to the pads to apply vibration or massage pressures to the user. The nesting apparatus 200 may also include the option of weight blankets 253 and eye masks to add additional comfort and compression when covering the body of the user thereby providing the user with an even greater sensation of nesting.

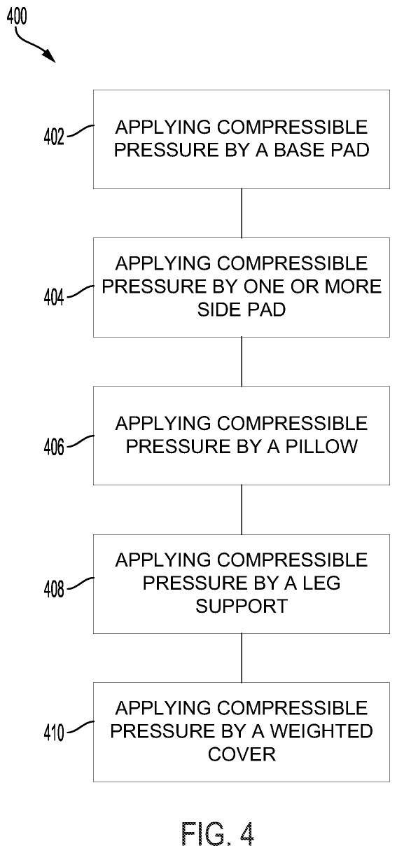

Referring now to FIG. 4, a flow chart depicting a method 400 of sensory intervention is depicted. The method 400, as described herein, may include one or more compressible pressures applied to a user by one or more components of a nesting device, such as those described above. According to one aspect, as shown in block 402, a compressible pressure may be applied by a base pad. Such a pressure may be applied by having a user lay on a base pad supported by a platform, or the like. The base pad may include a weight-absorbing, compressible material, such as a memory foam, gel, or the like, that cushions the user's body to provide a sense of weightlessness.

As shown in block 404, a compressible pressure may be applied to a user by one or more side pads. As described herein, the side pads may be coupled to one or more side supports slidably coupled to the platform to allow for a customizable size and customizable pressure applied to the user. The pads may include or be formed from a compressible material, such a memory foam, gel, or the like. According to one aspect the side pads may include one or more padded covers disposed over the side pads. The side pads and/or covers may apply a compressible pressure to the sides of the user's body. According to one aspect, the side pads and/or covers may define or form a pocket at or near the interface of the side supports and the platform. The pockets may be configured to receive the user's arms further enveloping the user's body and enhancing a burrowing sensation.

As shown in block 406, a compressible pressure may be applied by a pillow to the user's head and neck. The pillow may be, according to one aspect, contoured or shaped to optimally relieve stress and strain from the user's neck and head. The pillow may further be configured to raise the user's head to a height whereby the user's peripheral vision is minimally obstructed by the side pads, side covers, and/or side supports.

As shown in block 408, a compressible pressure may be applied by a leg support. According to one aspect, a leg support may be disposed on the base pad and underneath the user's knees to relieve stress and strain on the user's back and legs. Raising the knees and relieving the strain on the user's back and legs with the leg support may provide additional sensory intervention and enhance the feeling of weightlessness. The leg support may include or be formed from a compressible material, such as a memory foam, gel, or the like.

As shown in block 410, a compressible pressure may be applied by a weighted cover, such as a weighted blanket, to the user. The application of the weighted cover provides additional sensory intervention and forms a more-complete encapsulation of the user's body. According to one aspect, the application of compressible pressures to a significant portion of the user's body by the components of the nesting device creates a cocoon-like environment. In this environment, the user experiences a sensory intervention sufficient to allow the user to alter neural response, thereby improving biological, neurological, and psychological performance and well-being.

The foregoing disclosure and description of the invention is illustrative and explanatory of the embodiments of the invention. Various changes in the details of the illustrated embodiments can be made within the scope of the appended claims without departing from the true spirit of the invention. The embodiments of the present invention should only be limited by the following claims and their legal equivalents.

What is claimed is:

1. A nesting apparatus for supporting a user, the nesting apparatus comprising:
a platform,
a first side support, a second side support, and a headrest coupled to the platform;
a base pad disposed on the platform between the first and second side supports and the headrest;
first and second side pads respectively coupled to an inside surface of the first and second side supports,
a headrest pad coupled to an inside surface of the headrest;
wherein the headrest pad, first and second side pads and base pad are configured to apply a compressible pressure on a portion of the user's body.

2. The nesting apparatus of claim 1 further comprising a leg support disposed at an end of the base pad opposite the headrest, the leg support configured to support a user behind the user's knees.

3. The nesting apparatus of claim 1 further comprising a pillow disposed on the base pad at the headrest.

4. The nesting apparatus of claim 3 wherein the pillow is contoured to match a user's head and neck.

5. The nesting apparatus of claim 3 wherein the pillow includes a memory foam.

6. The nesting apparatus of claim 1 wherein the base pad includes a memory foam.

7. The nesting apparatus of claim 1 wherein the first and second side pads include a memory foam.

8. The nesting apparatus of claim 1 wherein the platform defines one or more slots at an end opposite the headrest.

9. The nesting apparatus of claim 1 wherein the platform is supported by two or more legs.

10. The nesting apparatus of claim 1 wherein the platform defines a hole in a central portion of the platform.

11. The nesting apparatus of claim 9 further comprising a netting disposed over the hole in the platform, the netting providing a limited yield under a weight of the user.

12. The nesting apparatus of claim 1 further comprising a weighted cover, the weighted cover applying a second compressible pressure on a second portion of the user's body.

13. The nesting apparatus of claim 1 wherein the first and second side pads are configured to form a first and second pocket near the coupling of the first and second side supports respectively, the first and second pockets each configured to receive a portion of the user's arms.

14. A method of sensory intervention, the method comprising:
applying a first compressible pressure on a first portion of a user's body by a base pad, the base pad supported by a platform.;
applying a second compressible pressure on a second portion of a user's body by first side pad;
applying a third compressible pressure on a third portion of a user's body by second side pad;
applying a fourth compressible pressure on a head and neck of a user's body by pillow;
wherein the first, second, third, and forth compressible pressures reduce sensory input on the user's body.

15. The method of claim 14 wherein the reduction of sensory input by the first, second, third, and forth compressible pressures alter neural response in the user.

16. The method of claim 14 further comprising applying a fifth compressible pressure under the user's legs.

17. The method of claim 16 wherein the fifth compressible pressure is applied under the user's knees with a leg support.

18. The method of claim 14 further comprising applying a weighted cover over the user's body, the weighted cover applying a covering compressible pressure.

19. A therapeutic device for sensory based interventions, the device comprising:
a platform,
a first padded side support, a second padded side support, and a padded headrest slidably coupled to the platform;
a base pad disposed on the platform between the first and second padded side supports and the headrest;
a headrest pad disposed at a first end of the base pad;
a leg support disposed at a second end of the base pad;
wherein the headrest pad, first and second padded side supports, the base pad, and leg support are configured to apply a compressible pressure on a portion of the user's body, thereby altering neural responses in the user.

20. The therapeutic device of claim 19 further comprising a weighted cover, the weighted cover configured to apply a second compressible pressure on a second portion of the user's body.
